(19) United States
(12) Patent Application Publication
(10) Pub. No.: US 2026/0114901 A1
(43) Pub. Date: Apr. 30, 2026

(54) ENDOSCOPIC METHOD

(71) Applicant: OLYMPUS CORPORATION, Tokyo (JP)

(72) Inventors: Junichi KOGISO, Hachioji-shi (JP); Takuma OKAMURA, Nagasaki-shi (JP)

(21) Appl. No.: 18/930,482

(22) Filed: Oct. 29, 2024

(51) Int. Cl. A61B 17/34; A61B 1/015; A61B 1/018; A61B 17/00; A61B 17/04; A61B 17/32; A61B 90/00; A61M 25/00

(52) U.S. Cl. CPC A61B 17/3478; A61B 1/015; A61B 1/018; A61B 17/0469; A61B 17/320016; A61B 2017/00269; A61B 2017/0034; A61B 2090/3983; A61M 2025/0089

(57) ABSTRACT

The endoscopic method includes compressing tissue surrounding a target site to form a compressed area and injecting a fluid into the tissue between the compressed area and the target site.

### TECHNICAL FILED

This disclosure relates to an endoscopic method.

### BACKGROUND

Conventionally, in endoscopic methods such as ESD (endoscopic submucosal dissection), a procedure has been used in which a local injection solution is injected into an appropriate area of the submucosa to bulge the target site of the treatment, making it easier to perform treatments such as incision and dissection on the target site of the treatment (Patent Document 1, etc.). In addition, a procedure has been used in which a block area is provided around the target site of the treatment to make it easier to perform treatment on the target site of the treatment (Patent Document 2, etc.).

### PRIOR ART DOCUMENTS

### Patent Documents

[Patent Document 1] JP 2003-153911 A
[Patent Document 2] U.S. Patent Publication No. 2019/0076283 Specification

### SUMMARY

An endoscopic method according to a first aspect of the present disclosure includes forming a compressed area by compressing tissues around a target site and injecting a fluid into the tissues between the compressed area and the target site.

An endoscopic method according to a second aspect of the present disclosure includes forming a crushed area by crushing the submucosa around a target site in the radial direction of the digestive tract and injecting a fluid into the tissues between the crushed area and the target site.

An endoscopic method according to a third aspect of the present disclosure includes forming a block area in tissues around a target site that blocks leakage of fluid and injecting a fluid into the tissues between the block area and the target site.

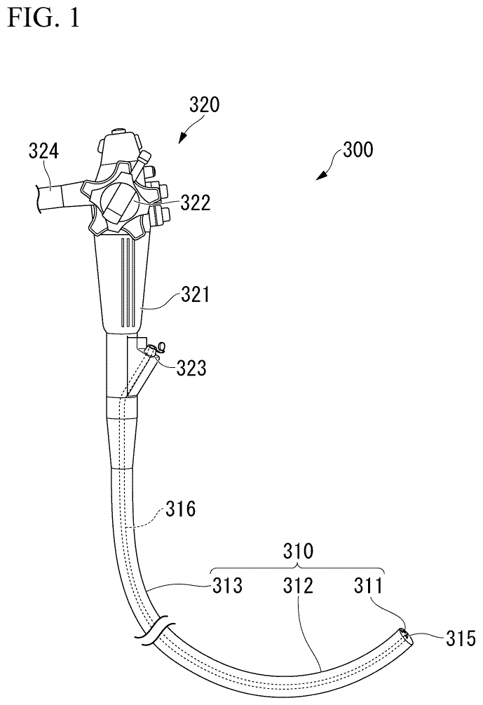

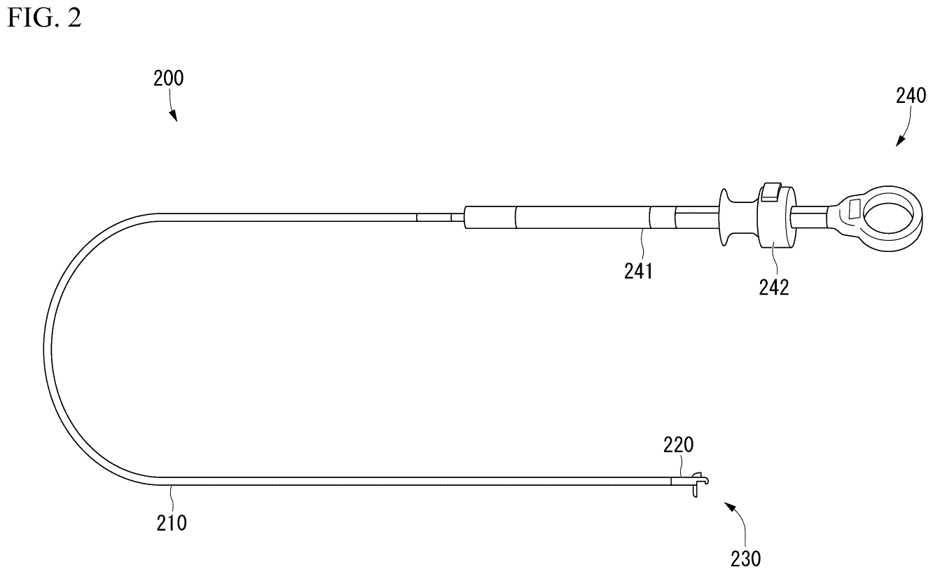

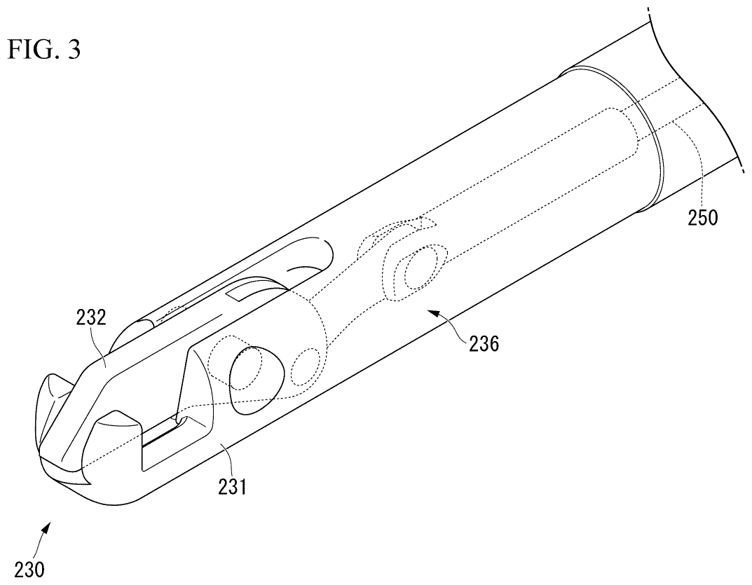

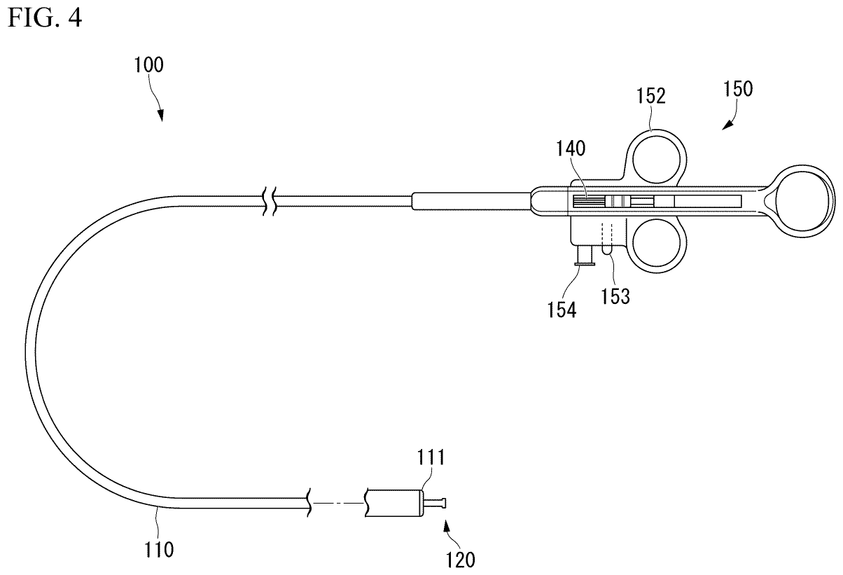

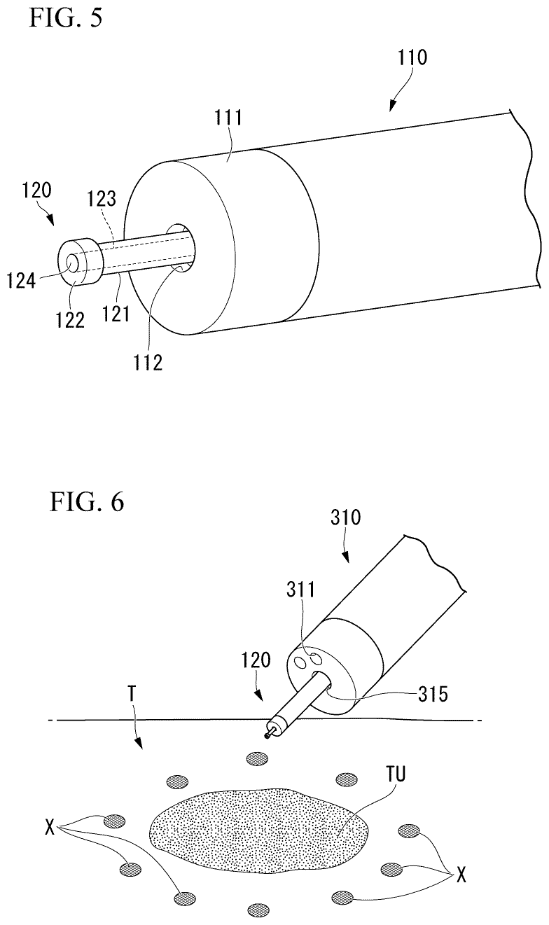

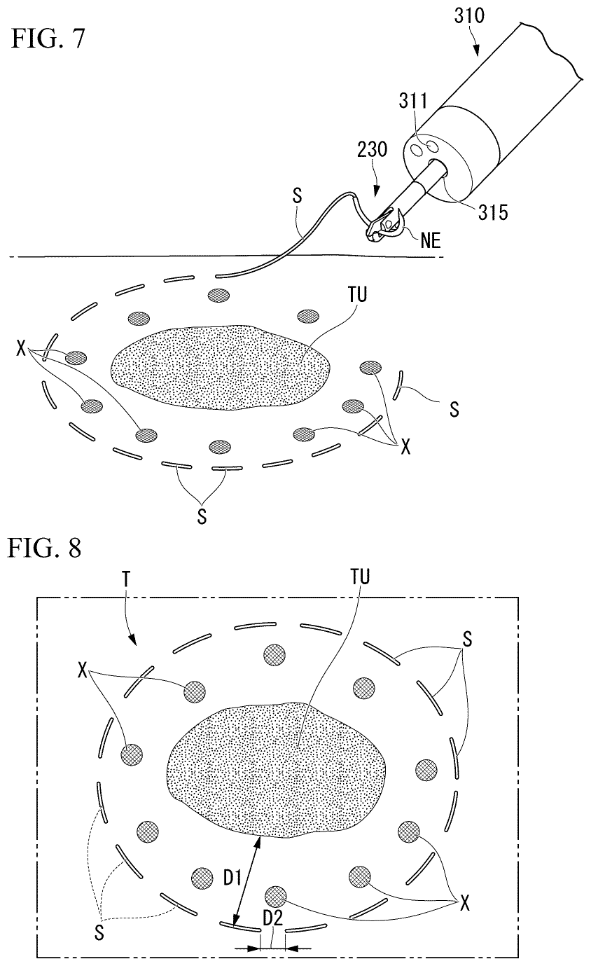

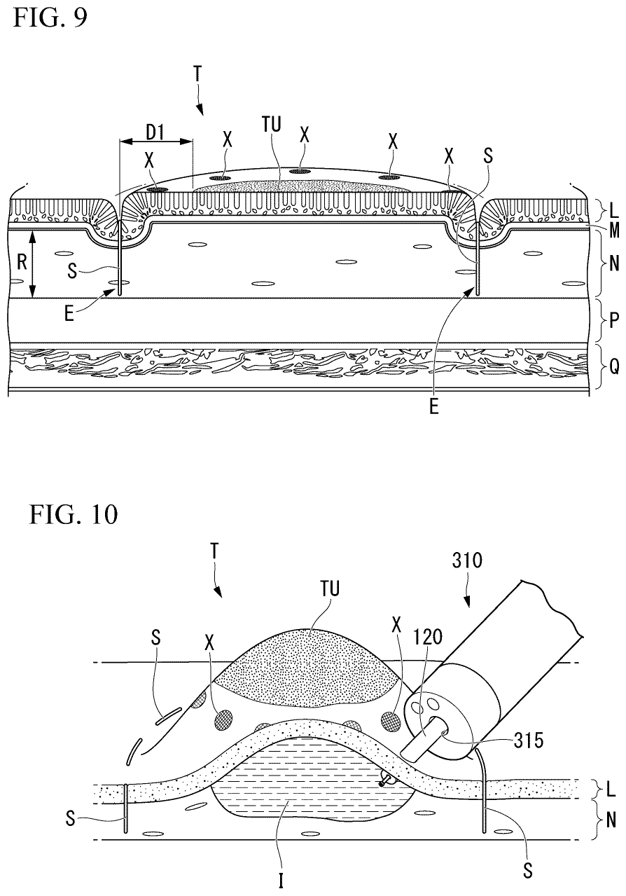

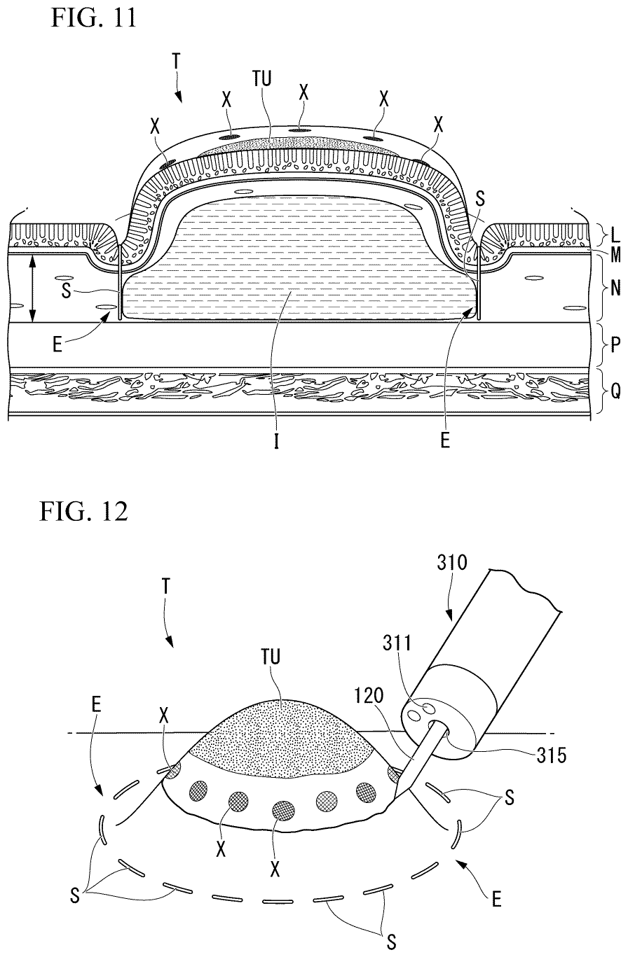

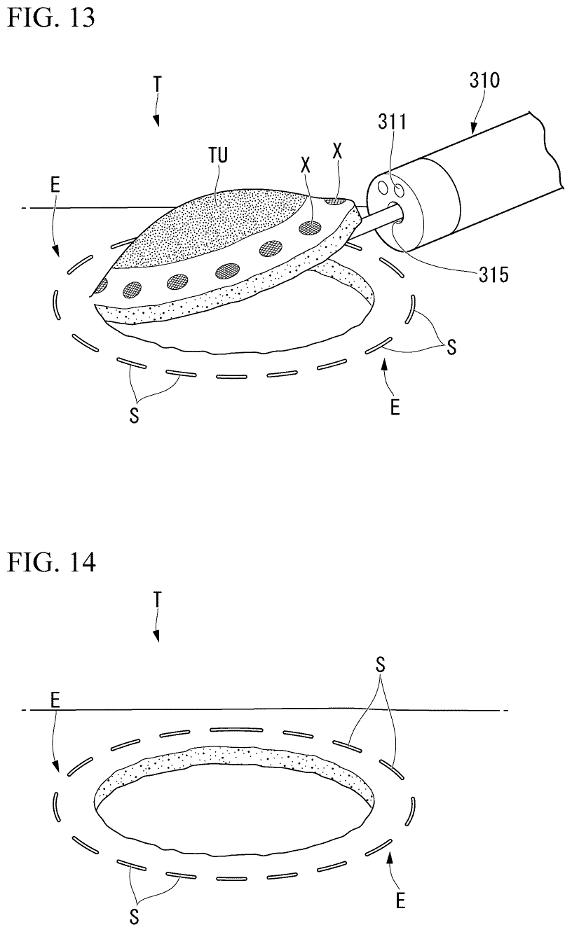

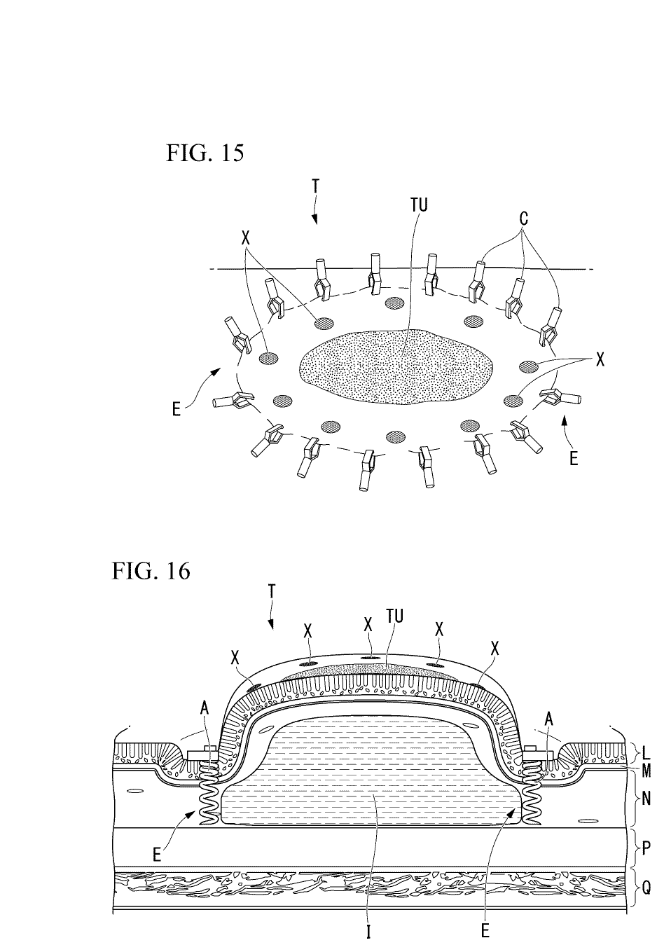

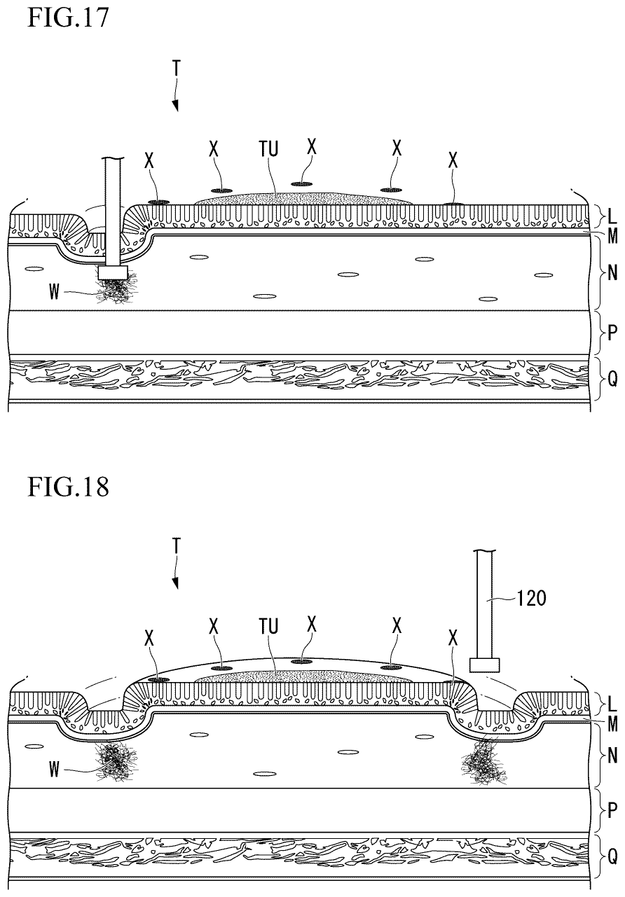

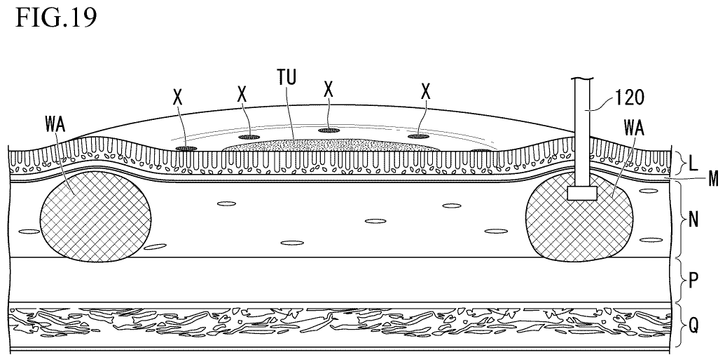

### BRIEF DESCRIPTION OF THE DRAWINGS

[FIG. 1] An overall view showing an endoscope used in endoscopic method according to a first embodiment.

[FIG. 2] An overall view showing a needle holder.

[FIG. 3] A perspective view showing a grasping portion of the needle holder.

[FIG. 4] An overall view showing a high-frequency knife.

[FIG. 5] A perspective view of the distal end of the high-frequency knife.

[FIG. 6] A view showing a marking step.

[FIG. 7] A view showing a suture step.

[FIG. 8] A view showing a sewn suture.

[FIG. 9] A cross-sectional view of a target site.

[FIG. 10] A view showing a local injection step.

[FIG. 11] A cross-sectional view of the target site after the local injection step.

[FIG. 12] A view showing an incision step.

[FIG. 13] A view showing a peeling step.

[FIG. 14] A view showing the target site after the peeling step.

[FIG. 15] A view showing a compressed area formed by a clip.

[FIG. 16] A view showing a compressed area formed by an anchor.

[FIG. 17] A view showing a cauterization step in endoscopic method according to a second embodiment.

[FIG. 18] A view showing the cauterization step.

[FIG. 19] A view showing a block area formed by local injection of a drug.

### DETAILED DESCRIPTION

### First Embodiment

An endoscopic method according to a first embodiment of the present invention will be described with reference to FIGS. 1 to 14. For endoscopic method according to this embodiment, an endoscope 300, a needle holder 200, and a high-frequency knife 100 are used.

FIG. 1 is an overall view of an endoscope 300.

The endoscope 300 is a known flexible endoscope and includes an insertion portion 310 that is inserted into the body from the distal end, and an operation portion 320 attached to the proximal end of the insertion portion 310.

The insertion portion 310 includes an imaging portion 311, a bending portion 312, and a flexible portion 313. Inside the insertion portion 310, a channel 316 is provided into which treatment tools such as the needle holder 200 and the high-frequency knife 100 are inserted. A distal end opening 315 of the channel 316 is provided at the distal end of the insertion portion 310.

The imaging portion 311 includes an imaging element such as a CCD or CMOS and is capable of capturing an image of the area to be treated (hereinafter also referred to as the “target site”). The bending portion 312 bends in response to the operation of the operation portion 320 by the operator. The flexible portion 313 is a flexible tubular portion.

The operation portion 320 is connected to the flexible portion 313. The operation portion 320 has a grasp 321, an input portion 322, a proximal end opening (forceps port) 323 of the channel 316, and a universal cord 324.

The grasp 321 is a portion that is held by the operator. The input portion 322 accepts an operation input for bending the bending portion 312. The universal cord 324 outputs the image captured by the imaging portion 311 to an image processing device.

FIG. 2 is an overall view of the needle holder 200.

The needle holder 200 includes a sheath 210, a rigid portion 220, a grasping portion 230, an operation portion 240, and an operation wire 250 that passes through the sheath 210. The needle holder 200 is used by inserting it into a channel 316 of an endoscope 300.

The sheath 210 is a long, flexible cylindrical member. The rigid portion 220 is provided at the distal end of the sheath 210. The operation portion 240 is provided at the proximal end of the sheath 210.

FIG. 3 is a perspective view showing the grasping portion 230 of the needle holder 200.

The grasping portion 230 includes a first grasping member 231, a second grasping member 232, and a link mechanism 236. The first grasping member 231 and the second grasping member 232 are configured to be capable of opening and closing.

The operation portion 240 has a main body 241 and a slider 242. The slider 242 is connected to the main body 241 so as to be capable of advancing and retracting. The operation wire 250 extending from the sheath 210 passes through the inside of the main body 241 and is connected to the slider 242.

The operation wire 250 is arranged inside the sheath 210 along the longitudinal axis of the sheath 210. The operator advances and retracts the slider 242 along the main body 241, causing the operation wire 250 to advance and retract. The grasping portion 230 opens and closes as the operation wire 250 advances and retracts.

FIG. 4 is an overall view of the high-frequency knife 100.

The high-frequency knife 100 is a high-frequency dual knife capable of, for example, water supply and incision, cauterization, and dissection. The high-frequency knife 100 includes a sheath 110, a knife 120, an operation wire 140, and an operation portion 150. The high-frequency knife 100 is used by being inserted into a channel 316 of an endoscope 300.

FIG. 5 is a perspective view of the distal end of the high-frequency knife 100.

The sheath 110 is a long cylindrical member that is flexible and insulating. An insulating distal end distal end 111 having a through hole 112 that penetrates in the longitudinal direction is attached to the distal end of the sheath 110.

The knife (electrode, rod) 120 is a long and slender member made of metal. The knife 120 is formed of a material such as stainless steel. The knife 120 includes a knife body 121 and a flange 122.

The knife body 121 is a round bar-shaped member made of metal. An operation wire 140 is attached to the proximal end of the knife body 121. When high-frequency current is supplied to the knife 120 from the operation wire 250, the knife body 121 and the flange 122 function as a monopolar electrode that outputs the high-frequency current to the biological tissue.

The flange (distal end portion) 122 is a circular plate-shaped conductive member provided at the distal end of the knife body 121. The radial width of the flange 122 perpendicular to the longitudinal direction is longer than the radial width of the knife body 121.

The knife body 121 and the flange 122 have a water supply pipe 123. The water supply pipe 123 is connected to a distal end opening 124 formed in the flange 122.

The operation wire 140 is a hollow coil wire that passes through the sheath 110. The distal end of the operation wire 140 is connected to the knife 120, and the proximal end of the operation wire 140 is connected to a slider 152 of the operation portion 150.

The operation portion 150 has an operation portion main body 151, a slider 152, a power supply connector 153, and a liquid supply port 154.

The slider 152 is attached so as to be movable along the operation portion main body 151. The proximal end of the operation wire 140 is attached to the slider 152. The operation wire 140 and the knife 120 advance and retract when the surgeon advances and retracts the slider 152 relative to the operation portion main body 151.

The power supply connector 153 is connectable to a high-frequency power supply device and is connected to the proximal end of the operation wire 140 via a conductive wire. The power supply connector 153 can supply high-frequency current supplied from a high-frequency power supply device to the knife 120 via the operation wire 140.

The fluid (medicinal liquid) supplied from the liquid supply port 154 passes through the hollow part of the operation wire 140 and the water supply pipe 123 of the knife 120 and is discharged from the distal end opening 124.

In place of the high-frequency knife 100, a high-frequency knife without a water supply function may be used. In that case, a treatment tool with a water supply function, such as a local injection needle with a water supply function, is used in combination.

Next, the endoscopic method according to this embodiment will be described. Specifically, the incision and dissection treatment of the lesion in ESD (endoscopic submucosal dissection) will be described.

As a preparation, the surgeon identifies the lesion using a known method. Specifically, the surgeon inserts the insertion portion 310 of the endoscope 300 into the digestive tract (e.g., esophagus, stomach, duodenum, large intestine) and identifies the lesion while observing the image obtained by the imaging portion 311 of the endoscope 300.

### <Marking Step>

FIG. 6 is a diagram showing the marking step.

The surgeon inserts the high-frequency knife 100 into the channel 316 and makes the knife 120 provided at the distal end of the high-frequency knife 100 protrude from the distal end opening 315 of the endoscope 300. The surgeon presses the knife 120 of the high-frequency knife 100 against the surrounding tissue surrounding the lesion TU, which is the target site T, to cauterize it, thereby applying a marking X. The marking X is applied by cauterizing the surface of the mucosal layer L, and the marking X does not reach the submucosal layer N, the mucosal basal layer M, or the muscular layer P (see FIG. 9).

### <Suture Step>

FIG. 7 is a diagram showing the suture step.

The surgeon removes the high-frequency knife 100 from the channel 316 of the endoscope 300 and inserts the needle holder 200 into the channel 316. The surgeon makes the grasping portion 230 of the needle holder 200 protrude from the distal end opening 315 of the endoscope 300. The surgeon uses the grasping portion 230 to grasp the suture needle NE to which the suture thread S is attached. The suture needle NE used in this embodiment is a curved needle. The treatment tool used in the suture step of the endoscopic method according to this embodiment is not limited to the needle holder 200, and may be another treatment tool capable of grasping the suture needle NE.

The surgeon sews the suture thread S to the surrounding tissue by suturing the surrounding tissue surrounding the lesion TU, which is the target site T, with the suture needle NE.

FIG. 8 is a diagram showing the sewn suture thread S.

The suture thread S is sewn to the outside of the marking X at a position that surrounds the periphery of the lesion TU. The distance DI from the cut portion of the lesion TU to the suture thread S is preferably 5 mm or more and 10 mm or less. This is to ensure sufficient space for the incision by the high-frequency knife 100 in the incision step.

The distance D2 of the stitches (the part exposed on the surface of the mucosal layer L) of the suture thread S when viewed from the mucosal surface is preferably 5 mm or less. The method of sewing the suture thread S may be backstitching so that the distance D2 is almost not generated. This is because if the distance D2 is large, the locally injected liquid (medicinal liquid) I is more likely to spread outward in the locally injected step.

FIG. 9 is a cross-sectional view of the target site T.

The surgeon moves the suture needle NE around the muscle layer P, drawing the mucosal layer L and the muscle layer P together with the suture thread S, and pinching and compressing the submucosal layer N to form a compressed area E. The compressed area E in the submucosal layer N is a part where the thickness (length in the radial direction R) of the digestive tract wall is smaller than the part not compressed by the suture thread S (for example, the thickness of the digestive tract wall at the position where the lesion TU is present). It is preferable that the suture thread S does not pass through the muscle layer P and reach the serosa Q.

In addition, the compressed area E in the submucosal layer N may be a crushed area where the submucosal layer N is crushed in the radial direction R of the digestive tract. The crushed area in the submucosal layer N is a part where the submucosal layer N is crushed and the length of the submucosal layer N in the radial direction R of the digestive tract is shorter than the part where the submucosal layer N is not crushed.

The surgeon does not necessarily have to move the suture needle NE around the muscle layer P. As long as the compressed area (crushed area) E can be sufficiently formed, the suture S does not need to reach the muscle layer P. It is sufficient that the suture S reaches the muscle layer P deep to the submucosal layer N.

### <Local Injection Step>

FIG. 10 shows the local injection step.

The surgeon removes the needle holder 200 from the channel 316 of the endoscope 300 and inserts the high-frequency knife 100 into the channel 316. The surgeon projects the knife 120 at the distal end of the high-frequency knife 100 from the distal end opening 315 of the endoscope 300.

The surgeon punctures and penetrates the surface of the mucosal layer L into which the local injection liquid (local injection liquid) I is to be injected with the knife 120. The area to be punctured with the knife 120 is, for example, the surface of the mucosal layer L between the compressed area E and the marking X. The surgeon inserts the distal end opening 124 of the distal end of the knife 120 into the submucosal layer N.

With the distal end opening 124 of the distal end of the knife 120 inserted in the submucosa N, the surgeon supplies the local injection liquid I to the fluid supply port 54 and injects the local injection liquid I into the submucosa N from the distal end opening 124.

FIG. 11 is a cross-sectional view of the target site T after the local injection step.

The surgeon injects the local injection liquid I from multiple locations to sufficiently bulge the lesion TU. Since there is a compressed area E around the area where the local injection liquid I is injected in the submucosa N, the local injection liquid I can be prevented from spreading outside the compressed area E. In other words, the area where the local injection liquid I is injected can be limited by the compressed area E. In the submucosa N, the local injection liquid can be suitably injected into the area where it is desired to inject the local injection liquid, and the tissue in the area where it is not desired to inject the local injection liquid I can be prevented from being stretched or destroyed by the local injection liquid I.

The local injection liquid I is, for example, physiological saline or hyaluronic acid. In order to sufficiently swell the lesion TU, the locally injected liquid I is preferably hyaluronic acid, which has a high viscosity. However, in the endoscopic method according to this embodiment, the compressed area E suppresses the spread of the locally injected liquid I, so even if the locally injected liquid I is physiological saline, the lesion TU can be sufficiently swelled.

### <Incision Step>

FIG. 12 is a diagram showing the incision step.

The surgeon moves the knife 120 while a high-frequency current is applied to the knife 120 to incise the entire circumference of the mucosal layer L around the lesion TU. The area to be incised with the knife 120 is, for example, the mucosal layer L between the compressed area E and the lesion TU, which is the target site T. The area to be incised with the knife 120 may be, for example, the mucosal layer L between the compressed area E and the marking X.

### <Dissection Step>

FIG. 13 is a diagram showing the dissection step.

With high-frequency current passing through the knife 120, the surgeon lifts up the incised flap-shaped mucosal layer L and cauterizes and dissects the submucosal layer N of the lesion TU.

FIG. 14 is a diagram showing the target site T after the dissection step.

The surgeon continues the above-mentioned operations (treatments) as necessary, and finally excises the lesion TU, completing the ESD procedure.

With the endoscopic method according to this embodiment, the area into which the local injection liquid I is injected can be limited by the compressed area E, and the local injection liquid I can be injected into the area intended by the surgeon. Furthermore, with the endoscopic method according to this embodiment, the lesion TU can be suitably bulged. Therefore, the imaging portion 311 makes it easier to view the dissection line from the front, and it is easier to secure space for incision by the high-frequency knife 100, so that safe incision and dissection can be achieved.

The first embodiment of the present invention has been described above in detail with reference to the drawings, but the specific configuration is not limited to this embodiment, and design modifications within the scope of the present invention are also included. In addition, the components shown in the above-mentioned embodiment and the following modified examples can be appropriately combined to form a configuration.

### Modification 1

In the above embodiment, the compressed area E is formed by a suture thread S. However, the method of forming the compressed area E is not limited to this. FIG. 15 is a diagram showing the compressed area E formed by a clip C. The surgeon clips the surrounding tissue surrounding the lesion TU, which is the target site T, with the clip C. The surgeon clamps and compresses the submucosal layer N with the clip C to form the compressed area E.

### Modification 2

In the above embodiment, the compressed area E is formed by a suture thread S. However, the method of forming the compressed area E is not limited to this. FIG. 16 is a diagram showing the compressed area E formed by an anchor A. The surgeon inserts the anchor A into the surrounding tissue surrounding the lesion TU, which is the target site T. The surgeon forms a compressed area E by pressing the submucosal layer N from the surface of the mucosal layer L with the anchor A. The anchor that forms the compressed area E is not limited to the screw-shaped anchor A exemplified in FIG. 16. The anchor that forms the compressed area E may be a T-shaped anchor.

### Second Embodiment

An endoscopic method according to a second embodiment of the present invention will be described with reference to FIG. 17 and FIG. 18. In the following description, the same reference numerals will be used to designate components that are common to those already described, and redundant description will be omitted.

An endoscopic method according to this embodiment will be described. Specifically, the incision and dissection procedure of the lesion in ESD (endoscopic submucosal dissection) will be described. The endoscopic method according to this embodiment forms a block area W instead of the compressed area E.

### <Marking Step>

Similar to the first embodiment, the surgeon presses the knife 120 of the high-frequency knife 100 against the surrounding tissue surrounding the lesion TU, which is the target site T, and cauterizes it to form a marking X.

### <Cauterization Step>

FIGS. 17 and 18 are diagrams showing the cauterization step.

The surgeon inserts the high-frequency knife 100 into the surrounding tissue surrounding the lesion TU, which is the target site T, and cauterizes the submucosal layer N. By cauterizing the submucosal layer N, the surgeon coagulates and solidifies the tissue to form a block area W.

The block area W can prevent the locally injected liquid I from leaking outside the block area W, similar to the compressed area E in the first embodiment.

The surgeon performs the locally injected step, incision step, and dissection step, similar to the first embodiment, to resect the lesion TU and complete the ESD procedure.

With the endoscopic method according to this embodiment, the area into which the locally injected liquid I is injected can be limited by the block area W, and the locally injected liquid I can be injected into the area intended by the surgeon. Furthermore, according to the endoscopic method of this embodiment, the lesion TU can be suitably bulged. Therefore, the imaging portion 311 makes it easier to view the dissection line from the front, and it is easier to secure space for incision by the high-frequency knife 100, so that safe incision and dissection can be achieved.

The second embodiment of the present invention has been described above in detail with reference to the drawings, but the specific configuration is not limited to this embodiment, and design changes and the like within the scope of the present invention are also included. In addition, the components shown in the above-mentioned embodiment and the modified examples shown below can be appropriately combined to configure the present invention.

### Modification 3

In the above embodiment, the block area W is formed by cauterizing the tissue. However, the method of forming the block area W is not limited to this. FIG. 19 is a diagram showing a block area WA formed by local injection of a drug (second drug solution). The surgeon forms the block area WA by injecting a drug into the submucosal layer N of the surrounding tissue surrounding the lesion TU, which is the target site T. The drug for forming the block area WA is ethanol, etc. The drug is injected by a high-frequency knife 100, a treatment tool with an injection needle, etc. The block area WA blocks the locally injected liquid I, and can suppress the locally injected liquid I from leaking out of the block area W.

What is claimed is:

1. An endoscopic method, comprising:
forming a compressed area by compressing tissue surrounding a target site; and
injecting a fluid into an interior of the tissue between the compressed area and the target site.

2. The endoscopic method according to claim 1, further comprising:
applying a marking on a surface of a mucosal layer surrounding the target site before forming the compressed area.

3. The endoscopic method according to claim 1, further comprising:
incising the tissue between the compressed area and the target site.

4. The endoscopic method according to claim 2, wherein
the injecting of the fluid includes injecting the fluid into the interior of the tissue from a surface of the tissue between the compressed area and the marking.

5. The endoscopic method according claim 2, further comprising:
incising the tissue between the compressed area and the marking.

6. The endoscopic method according to claim 5, wherein
the incising includes incising the tissue around the marking over an entire circumference.

7. The endoscopic method according to claim 1, wherein the fluid is injected from a surface of a mucosal layer into a submucosal layer.

8. The endoscopic method according to claim 1, wherein
the compressed area is formed by pinching a submucosal layer from a radial direction of a digestive tract.

9. The endoscopic method according to claim 1, wherein
the compressed area is formed by pressing from a surface of a mucosal layer.

10. The endoscopic method according to claim 1, wherein
the compressed area is formed by tightening a suture thread that has been passed from a surface of a mucosal layer to a muscular layer.

11. The endoscopic method according to claim 1, wherein
the compressed area is formed by clipping a mucosal layer, a submucosal layer, and a muscular layer.

12. The endoscopic method according to claim 1, wherein
the compressed area is formed by driving an anchor from a surface of a mucosal layer to a muscle layer.

13. An endoscopic method, comprising:
forming a crushed area by crushing a submucosal layer around the target site in the radial direction of the digestive tract; and
injecting a fluid into the tissue between the crushed area and the target site.

14. The endoscopic method according to claim 13, further comprising:
applying a marking on a surface of a mucosal layer around the target site before forming the crushed area.

15. The endoscopic method according to claim 13, further comprising:
incising the tissue between the crushed area and the target site.

16. The endoscopic method according to claim 14, wherein
the injecting of the fluid includes injecting a fluid into the submucosal layer from a surface of the mucosal layer between the crushed area and the marking.

17. The endoscopic method according to claim 14, further comprising:
incising a mucosal layer between a contact area and the marking.

18. An endoscopic method, comprising:
forming a block area in a tissue surrounding a target site to block leakage of fluid; and
injecting the fluid into tissue between the block area and the target site.

19. The endoscopic method according to claim 18, wherein
the block area is formed by cauterizing the tissue surrounding the target site.

20. The endoscopic method according to claim 18, wherein
the block area is formed by injecting a drug.
